(19) United States
(12) Patent Application Publication
(10) Pub. No.: US 2014/0050724 A1
(43) Pub. Date: Feb. 20, 2014

(54) Compositions and Methods

(71) Applicants: Hemant K. Roy, Highland Park, IL (US); Ramesh K. Wali, Darien, IL (US); Dhananjay Kunte, Vernon Hills, IL (US)

(72) Inventors: Hemant K. Roy, Highland Park, IL (US); Ramesh K. Wali, Darien, IL (US); Dhananjay Kunte, Vernon Hills, IL (US)

(73) Assignee: NORTHSHORE UNIVERSITY HEALTHSYSTEM, Evanston, IL (US)

(21) Appl. No.: 14/114,205

(22) PCT Filed: Apr. 27, 2012
(86) PCT No.: PCT/US2012/035434

§ 371 Date: Oct. 26, 2013

Related U.S. Application Data
(60) Provisional application No. 61/479,493, filed on Apr. 27, 2011.

(51) Int. Cl. A61K 31/08; A61K 45/06

(52) U.S. Cl. CPC A61K 31/08; A61K 45/06
USPC 424/133.1; 514/723; 568/679

(57) ABSTRACT

Compositions comprising polyethylene glycol (PEG) are disclosed for the prophylaxis and/or treatment of head and neck squamous cell carcinomas (HNSCC). Methods for the prophylaxis and/or treatment of HNSCC comprising administering an effective amount of PEG are also disclosed. Also disclosed are methods and compositions for suppressing the surface expression of EGFR using PEG.

The present invention concerns compositions for use in the prophylaxis and/or treatment of squamous cell carcinomas such as head and neck squamous cell carcinoma (HNSCC). The present invention also concerns methods for preventing and/or treating such carcinomas. Other aspects, objects and advantages of the present invention will be apparent from the description below.

### BACKGROUND OF THE INVENTION

Head and neck cancer is estimated to have caused approximately 7900 deaths in the U.S. in 2010 (Pfister D. G et al; J. Natl. Compr. Canc. Netw. 2011; 9:596-650). Common risk factors for developing this disease include the smoking or chewing of tobacco, consumption of alcohol, chewing of betel nut and/or infection with human papillomavirus (HPV).

Head and neck squamous cell carcinoma accounts for approximately 3 percent of all cancers in the United States. As used herein, the term “HNSCC” includes any cancer of the head and neck that begins in squamous cells (thin, flat cells that form the surface of the skin, eyes, various internal organs, and the lining of hollow organs and ducts of some glands).

HNSCC is associated with high rate of death and morbidity. This aggressive epithelial malignancy implicates the mucosal lining of the upper aerodigestive track including the oral cavity, oropharynx and larynx.

Even following curative treatment, patients still have an increased lifetime risk of developing new second cancers, usually in the head and neck or lung regions. An early risk factor for developing HNSCC is oral leukoplakia, while approximately 30% of survivors develop second malignancies.

Even those patients who do not experience recurrence of the primary cancer have a high risk of developing a second malignancy as a result of antecedent premalignant cancer field, also known as “field cancerization”.

Chemoprevention of HNSCC with, for example, Nonsteroidal anti-inflammatory drugs (NSAIDS), retinoic acid, beta-carotene have to-date demonstrated minimal efficacy and are marred with toxicity. Despite extensive treatment options, the 5-year survival rate for HNSCC has not greatly improved over the last 4 to 5 decades. Furthermore, as a consequence of the disease severity and the ensuing radical surgical management, HNSCC patients have a worsening quality of life due to debilitating changes in facial appearance, speech, swallowing and breathing.

As such, there still exists a need for improving the prevention and/or treatment of this disease.

### SUMMARY OF THE INVENTION

The present invention is based, at least in part, on an observation that polyethylene glycol (PEG) exhibits antiproliferative activity in HNSCC.

In accordance with the present invention there is provided a method for preventing and/or treating squamous cell carcinoma, particularly HNSCC in a subject, the method comprising administering to the subject an effective amount of polyethylene glycol (PEG). Compositions for use in such a method are also provided.

Another embodiment, the present invention provides a method for preventing and/or treating HNSCC in a subject comprising administering to the subject an effective amount of PEG, wherein the PEG suitable for use in the method preferably has an average molecular weight between about 1000 daltons and 20,000 daltons, preferably between about 2000 daltons and 9,000 daltons, more preferably between about 3000 and 8000, most preferably about 3350, 4000 or 8,000.

In another embodiment, the invention provides a composition for use in the prophylaxis and/or treatment of HNSCC as described in various aspects and embodiments of the invention herein, the composition comprising (e.g. as its sole therapeutically active constituent) an effective amount of PEG. The composition may comprise PEG having an average molecular weight as described herein.

In an embodiment, the present invention provides a method for preventing and/or treating HNSCC in a subject comprising topically administering to said subject an effective amount of PEG. Compositions for use in such a method are also provided.

In accordance with another embodiment, the present invention provides a method for reducing or suppressing HNSCC initiation and/or proliferation in a subject comprising topically administering to the region of the subject afflicted with HNSCC and/or a pre-malignant lesion and/or oral leukoplakia, an effective amount of PEG. Compositions for use in such a method are also provided.

In accordance with another embodiment, the present invention provides a method for preventing and/or treating HNSCC in a subject comprising locoregionally administering to the subject an effective amount of PEG. Compositions for use in such a method are also provided.

The present invention further provides a composition for locoregional use in the prevention and/or treatment of HNSCC in a subject afflicted with said disease wherein the composition comprises an effective amount of PEG.

It is further contemplated that in an embodiment of the present invention, a method for preventing and/or treating HNSCC in a subject, wherein the effective amount of PEG is administered to the subject from 1 to 5 time(s) a day, preferably 2 to 4 times a day, more preferably 3 times a day. Compositions for use in such a method are also provided.

In an embodiment, the present invention provides a method for reducing or suppressing HNSCC proliferation in the head and neck of a subject comprising administering to the subject an effective amount of PEG to one or more of: the lip, oral cavity (including the tongue, buccal mucosa, alveolar ridge, retromolar trigone, gums, floor of mouth, hard palate), salivary glands, nasal cavity (including nasopharynx), paranasal sinuses, pharynx (including oropharnyx such as the base of tongue, soft palate, tonsillar pillar and fossa) hypopharnyx (including pyriform sinus, lateral pharyngeal wall, posterior pharyngeal wall, postcricoid pharynx), and larynx (including supraglottis (e.g. false cords, arytenoids, epiglottis, arytenoepiflottic fold), glottis, subglottis) of the subject.

The present invention further provides methods for preventing and/or treating HNSCC in a subject as described in various aspects and embodiments of the invention herein, wherein the HNSCC has afflicted one or more of: the lip, oral cavity (including the tongue, buccal mucosa, alveolar ridge, retromolar trigone, gums, floor of mouth, hard palate), salivary glands, nasal cavity (including nasopharynx), paranasal sinuses, pharynx (including oropharnyx such as the base of tongue, soft palate, tonsillar pillar and fossa) hypopharnyx (including pyriform sinus, lateral pharyngeal wall, posterior pharyngeal wall, postcricoid pharynx), and larynx (including supraglottis (e.g. false cords, arytenoids, epiglottis, arytenoepiflottic fold), glottis, subglottis) of the subject, the method comprising administering to the afflicted area of the subject, an effective amount of PEG. Compositions for use in such methods are also provided.

A preferred embodiment of the invention is a method for preventing and/or treating HNSCC in a subject wherein the HNSCC has afflicted the oral cavity (or anatomical site thereof) comprising administering (for example locoregionally administering) to the oral cavity an effective amount of PEG. Compositions for use in such a method are also provided.

In accordance with a further embodiment, the present invention provides a method for reducing or inhibiting Epidermal Growth Factor Receptor (EGFR) surface expression and/or phosphorylation of the receptor in the squamous cells of the head and neck of a subject, the method comprising administering to the subject an effective amount of PEG. Compositions for use in such a method are also provided.

In another embodiment, the present invention comprises a method for preventing and/or treating HNSCC in a subject which method comprises co-administering to the subject an effective amount of PEG with an effective amount of one or more additional therapeutic agent(s). Compositions for use in this method comprising an effective amount of PEG, optionally further comprising the one or more additional therapeutic agent(s) are also provided.

In another aspect of the invention there is provided a method for preventing and/or treating HNSCC in a subject which method comprises administering to said subject:
(a) an effective amount of a therapeutic agent such as an anti-EGFR agent (for example an anti-EGFR antibody such as cetixumab);
(b) an effective amount of PEG.

In some embodiments of this aspect of the invention, step (a) occurs before step (b). In other embodiments of this aspect of the invention, step (a) occurs after step (b). In further embodiments of this aspect of the invention, step (a) and step (b) occur concurrently.

In another embodiment of the invention, there is provided a method for preventing and/or treating HNSCC in a subject in remission of HNSCC, the method comprising administering to the subject an effective amount of PEG. The subject may be in partial or complete remission. Compositions for use in such a method are also provided.

In another embodiment of the invention there is provided a method for ameliorating (such as preventing) the recurrence of HNSCC in a subject in remission of that disease, the method comprising administering to the subject an effective amount of PEG. Compositions for use in such a method are also provided.

In another embodiment of the invention there is provided a method for preventing and/or treating metastatic HNSCC in a subject which method comprises administering to the subject an effective amount of PEG. Compositions for use in such a method are also provided.

In another embodiment of the invention there is provided a method for preventing and/or treating locally advanced HNSCC in a subject which method comprises administering to the subject an effective amount of PEG. Compositions for use in such a method are also provided.

In another aspect of the invention there is provided a method for regressing a squamous cell carcinoma in a subject which method comprises (a) administering an effective amount of PEG. Compositions for use in such a method, such as described herein, are also provided. In some embodiments of this aspect of the invention, the squamous cell carcinoma is HNSCC. Preferably, the squamous cell carcinoma over-expresses EGFR. The EGFR expression status of the squamous cell carcinoma may be determined according to standard methods, techniques and kits such as described herein.

In an embodiment of this aspect of the invention, the method further comprises (b) resecting and/or ablating the squamous cell carcinoma and/or administering an effective amount of a therapeutic agent. In this embodiment of the invention, step (b) may occur after step (a). By regressing the squamous cell carcinoma, the present invention may reduce the degree of trauma or treatment related adverse events to the subject resulting from subsequent therapeutic procedures.

In another aspect of the invention, there is provided a method for preventing and/or treating HNSCC in a subject comprising;
(a) resecting and/or ablating one or more HNSCC carcinomas;
(b) administering to the subject an effective amount of PEG.

In one embodiment of this aspect of the invention, step (a) occurs before step (b). In another embodiment, step (b) occurs before step (a).

The invention further provides the use of an effective amount of PEG in the manufacture of a medicament, for example the compositions described herein, for the treatment and/or prophylaxis of HNSCC.

The invention further provides the use of an effective amount of PEG in the manufacture of a medicament, for example the compositions described herein, for regressing a squamous cell carcinoma such as HNSCC.

### DESCRIPTION OF THE FIGURES

FIG. 1: A graph depicting the antiproliferative activity of PEG 8000 in SCC-25 cells in vitro, a model for HNSCC. SCC-25 cells were seeded in a 96-well plate and then treated with different concentrations of PEG 8000 (0.62 mM (0.5% w/v) to 12.5 mM (10% w/v) for 24 h. Changes in the cellular proliferation (cell number) were assayed by using a standard WST-1 assay (Roche Diagnostics, Indianapolis, Ind.). The experiments were performed in triplicate for 3 determinations. The data are presented as mean±S.D.; [symbol] p<0.05, ★p<0.001 compared to vehicle alone.

FIG. 2: A graph depicting the effects of PEG 3350 or 8000 on the levels of membrane (surface) epidermal growth factor receptor (EGFR) expression in SCC-9 and SCC-25 cells. SCC-9 and SCC-25 cells were treated with a 5% w/v solution of PEG 3350, or with a 5% w/v solution of PEG 8000. The cells were trypsinized, stained for surface expression of EGFR and subjected to flow cytometric analysis. The experiments were performed in triplicate for 3 determinations. The data are presented as mean±S.D.; ^{x}p<0.01, *p<0.001 compared to corresponding control.

FIG. 3: A graph depicting the results of in vivo topical administration of PEG-8000 on the inhibition of tumor progression in the rat 4-Nitroquinoline 1-oxide (4NQO) model of head and neck squamous cell cancer. To develop this in vivo model, rats were given ad libitum access to 4NQO (20 ppm)-supplemented water. After 14 weeks, the rats were switched to regular water and randomized equally into PEG or vehicle-treated control groups. The rats either received a daily (3 to 4 minute) topical application of 10% PEG 8000 or PBS (vehicle control) in the oral cavity (tongue, buccal floor/roof). The rats were euthanized after 14 weeks; tongues were excised and subjected to macroscopic assessment of tumor burden, assessing both tumor number (FIG. 3 upper panel) and tumor volume (FIG. 3 lower panel). The data are presented as mean±S.D.

FIG. 4: A series of photographs comparing the palates and tongues of animals exposed to 4NQO only (top panel, left to right), or both 4NQO and PEG 8000 (bottom panel, left to right). The photographs reflect the reduced tumor volume in PEG 8000 treated tissue compared to vehicle treated rats as depicted in FIG. 3.

FIG. 5: A series of photomicrographs of immunohistochemically stained sections of 4NQO treated rat tongue showing the effect of oral topical application of PEG 8000 on the premalignant epithelial hyperproliferation. To evaluate the histopathological grading of the oral/tongue tissue, the formalin-fixed and H&E stained samples were subjected to pathological evaluation. As shown (upper panel), the H&E stained sections from 4NQO-treated rats showed a number of localized regions of mild-to-moderate epithelial dysplasia that was normalized in PEG 8000 treated rats. To further study the effect of PEG 8000 application on the mucosal proliferation in the tongue/oral region of 4NQO-treated rats, the tissue sections were subjected to immunohistochemical analyses of the nuclear antigen Ki67, a well-defined marker of proliferation (lower panel). As shown, compared to carcinogen-free control, 4NQO-treatment increased the proliferation in the tongue mucosa as depicted by an increased number of Ki67-labelled cells/optical field (p<0.01) compared to control. However, when the carcinogen-initiated rats were subjected to topical application of PEG 8000, a reduction in the number of Ki-67 positive cells was observed compared to the 4NQO group (p<0.01). In the carcinogen treated group, a higher percentage of cells were found to be Ki-67 positive in the supra-basal compartment of the stratified squamous tongue epithelium, when compared to control. PEG 8000 treatment reduced the number of Ki-67 positive cells in the supra-basal compartment, restricting expression mostly to the basal layer as seen in the control group.

FIG. 6: A photomicrograph depicting the effect of topical application of PEG 8000 on the expression of EGFR. The formalin-fixed tongue sections from control (carcinogen-untreated) and PEG-8000 treated or untreated 4NQO-rats were subjected to immunohistochemical assessment of EGFR expression. As depicted, baseline EGFR expression in the tongue mucosa of 4NQO rats was significantly higher than that of control (non-carcinogen treated) rats (p<0.00001). Topical application of PEG 8000 to 4NQO rats, however, caused a significant reduction in the expression of EGFR compared to PEG-untreated 4NQO-rats (p <0.005). The control and 4NQO-PEG sections have little or no staining for EGFR, whereas the 4NQO section is heavily stained for EGFR.

FIGS. 7, 8 and 9: Graphs depicting reduced cellular proliferation and EGFR expression by different PEG formulations (PEG 3350, PEG 4000 and PEG 8000) in SCC-25 cells. The cells were seeded either in 96-well plates (WST-1 assay) or 60 mm cell culture dishes (EGFR and PCNA expression). Cells were treated separately with PEG 3350 (FIG. 7), PEG 4000 (FIG. 8) and PEG 8000 (FIG. 9) for 24 h. The cellular proliferation was determined by both WST-1 assay and measuring the cellular proliferation marker Proliferating Cell Nuclear Antigen (PCNA) by Western blotting. EGFR expression was also determined by Western blotting. All the experiments were performed in triplicate. For WST-1 assay each experiment had 12 determinations, while for EGFR and PCNA measurements there were 3 determinations each. The data are presented as mean percent of control (no PEG treatment) for the respective biomarkers (WST-1, EGFR, and PCNA)±S.E.M.; *p<0.05 and ^{X}p<0.0001.

FIG. 10: A graph depicting the inhibition of surface expression of EGFR by different PEG formulations (PEG 3350, PEG 4000 and PEG 8000) in SCC-9 cells. For these studies, SCC-9 cells were treated with 10% w/v PEG for 24 h and subjected to flow cytometric analysis to determine the expression of surface EGFR. The histograms are presented as percent of control (no PEG treatment) for the surface expression of EGFR. The experiments were performed in triplicate with 4 determinations each. The data are presented as mean percent of control (no PEG treatment)±S.E.M.; *p<0.0001.

FIGS. 11 and 12: Graphs depicting the effect of PEG 8000 on proliferation and EGFR expression in human papillomavirus (HPV) (−) and HPV (+) cells. To study the role of HPV status on the effect of PEG 8000 on EGFR expression and proliferation, HPV (−) OKF 6 cells (FIG. 11) or HPV (+) HOK cells (FIG. 12) were treated with different concentrations of PEG 8000 for 24 h. The cellular proliferation was determined by both WST-1 assay and measuring the cellular proliferation marker PCNA by Western blotting. EGFR expression was also determined by Western blotting. All the experiments were performed in triplicate. For WST-1 assay each experiment had 12 determinations, while for EGFR and PCNA measurements there were 3 determinations each. The data are presented as mean percent of control (no PEG treatment) for the respective biomarkers (WST-1, EGFR, and PCNA)±S.E.M.; *p<0.05 and ^{x}p<0.0001.

FIGS. 13, 14 and 15: In order to study the effect of PEG on the regression of tumor growth, an orthotopic model was utilized, in which approximately 1 million SCC-25 cells were directly implanted near the tongue region of athymic mice. Two weeks later the mice were randomly divided into two groups of 12 animals each and subjected to daily oral topical application of 10% w/v PEG 8000 or PBS using a Q-tip. The mice were euthanized 19 to 20 days post treatment and tumors excised and weighed. PEG-8000 caused a significant decrease in the tumor weight compared to control (29.8%; *p<0.05, FIG. 13). Also evaluated were the Ki-67 proliferation marker and EGFR immunostaining in the tongue sections. PEG 8000 significantly reduced the proliferative index (31.5%; *p<0.002, FIG. 14) and EGFR staining (42.4%; *p<0.005, FIG. 15) in the PEG-treated groups compared to control. The data are presented as mean±S.E.M.

### DETAILED DESCRIPTION OF THE INVENTION

Compositions of the Invention.

Compositions of the invention may comprise an effective amount of PEG as its sole therapeutically active constituent or may contain one or more therapeutic agents (particularly anti-cancer agents) as described in more detail below. The term “polyethylene glycol” (PEG), otherwise known as poly(oxyethylene) or poly(ethylene oxide) (PEO), refers to a polymer of ethylene oxide. The polyethylene glycol (PEG) used in the invention typically has the general formula H—(OCH₂CH₂)ₙ OH, although other polyethylene glycol compounds may be used such as end-capped structures and polyoxyethylenes that include minor amounts of alkylene oxide units other than ethylene oxide.

The polyethylene glycol (PEG) used in compositions of the invention may have an average molecular weight (for example a weight average molecular weight), in a range wherein the lower limit of the range is selected from the group consisting of: 1000, 2000, 3000, 4000, 6000; and an upper limit of the range is, selected independently, from the group consisting of: 3000, 4000, 5000, 6000, 7000, 8000, 9000, 10,000, 12,000, 15,000, 20,000. Preferred ranges are wherein the lower limit is 3000 or 4000 and the upper limit is, selected independently, 5000, 6000, 7000, 8000 or 9000.

For example, the PEG may be PEG 3350, PEG 4000 or PEG 8000 as defined in some national or regional pharmacopias. Further examples of suitable PEGs recognized in some national or regional pharmacopoias include Macrogols, for example Macrogol 3350, Macrogol 4000, Macrogol 8000. Preferably, the PEG is not systemically absorbed to any significant extent when topically administered to the subject.

Compositions of the present invention may further comprise other constituents such as another therapeutic agent (see below), and one or more excipients. Examples of excipients include one or more electrolytes such as sodium chloride, potassium chloride, sodium bicarbonate, sulphate such as sodium sulphate. In one embodiment, compositions of the invention comprise sodium chloride and potassium chloride and optionally sodium bicarbonate. Compositions of the invention may comprise one or more sweetener(s) (such as aspartame, acesulfame potassium (acesulfame K), sucralose and saccharine and combinations thereof) and one or more flavouring(s) (such as orange, lemon-lime, lemon, citrus, chocolate, tropical fruit, aloe vera, tea, strawberry, grapefruit, blackcurrant, pineapple and vanilla). Compositions may further comprise ascorbate and/or citrate. Compositions of the invention may further comprise preservatives and other additives such as, for example, antimicrobials, anti-oxidants, carriers, chelating agents, and inert gases and the like as known and called for by acceptable galenic practice. Preferably, the carrier is a pharmaceutically acceptable carrier. The carrier can be any of those conventionally used and is limited only by chemico-physical considerations, such as solubility and lack of reactivity with the PEG and/or other therapeutic agents (if present) and by the route of administration. Pharmaceutically acceptable carriers are well-known to those skilled in the art and are readily available. It is preferred that the pharmaceutically acceptable carrier be one which is chemically inert to the active agent(s) and one which has no detrimental side effects or toxicity under the conditions of use.

Compositions of the invention may be presented in a variety of preparations. For example, compositions of the invention may be prepared in the form of an aqueous solution or suspension (for example a mouthwash), emulsion, paste (e.g. toothpaste), cream, balm (e.g. lip balm), ointment, foam, paint, sponge, gel, chewing gum, spray, lozenge, troche, syrup (e.g. viscous syrup), linctus, slurry, film (e.g. orodispersible film), tablet (e.g. orodispersible tablet), capsule (e.g. liquid-gel capsule), granule, caplet, buccal patch. Particularly preferred preparations of compositions of the invention include solutions, suspensions, linctus and paints.

Depending on the intended use of the composition of the invention, certain preparations may be particularly apt. For example, where it is desired to administer a composition of the invention to the lip, the composition may be prepared as a lip balm or cream. Where it is desired to administer the composition to an area of the buccal mucosa, a mouthwash, spray or lozenge may be particularly appropriate. Where the preparation is a mouthwash, it maybe gargled or swirled around the mouth before swallowing or expelling. Compositions of the invention may be prepared as an aerosol preparation, particularly for nasal, buccal and oropharynx administration. Compositions of the invention may also be prepared as non-pressured preparations, such as in a nebulizer or an atomizer. In some embodiments, the composition of the invention may be a linctus preparation, a preparation form well known to those skilled in the art. This form may assist in increasing the contact time between PEG and the target tissue (such as the pharynx and/or larynx mucosa) and may be particularly appropriate in the prophylaxis and/or treatment of HNSCC at these anatomical sites.

In one embodiment, compositions of the invention are administered topically to the subject. Topical administration, in the context of the present invention, refers to the application of a composition of the invention to a surface of the subject's body. Topical administration includes application to an internal surface of the subject, for example the buccal mucosa. Compositions of the present invention may be administered to the target area (e.g. the carcinoma) and in the local region thereof (sometimes referred to herein and in the art as “locoregional administration”). Typically, compositions of the present invention are applied to a region of the carcinoma that is accessible at the surface of subject's body, for example, surface exposed HNSCC that has afflicted the lip, tongue, buccal mucosa (e.g. buccal floor and/or roof), nasal cavity, pharynx, larynx and anatomical sites thereof. Topical administration may include per os administration of compositions of the invention. For example, topical administration may include swallowing a composition of the invention. Accordingly, contact between the composition of the invention and the surface exposed region of the carcinoma (if present) is enabled as the composition passes into the stomach via the pharynx.

An effective amount of PEG to be employed therapeutically will depend, for example, upon the therapeutic and treatment objectives (e.g. prophylaxis or treatment or both), the route of administration, the age, body mass, condition of the subject undergoing treatment or therapy (for example, by determining the subject's performance status), stage and/or aggressiveness (e.g. TNM score) of the carcinoma (if present), any auxiliary or adjuvant therapies being provided to the subject, and on the subjects previous response (if appropriate) to therapy with compositions of the invention. An effective amount of PEG may be determined, at least in part, by the desired reduction in EGFR surface expression in the target tissue (for example, a HNSCC carcinoma). An effective amount of PEG, according to the present invention, may produce in the target tissue a reduction in EGFR expression of at least 30% (or thereabout), for example, at least 40%, compared to the expression observed prior to contact with PEG.

Reduction of EGFR surface expression in a target tissue such as a carcinoma may be influenced by the contact time between PEG and the target tissue. In general, a greater reduction in EGFR surface expression in the target tissue may be observed with increased contact time. For example, contact time between PEG and the target tissue may be from 5 seconds to 10 minutes, e.g. 1 to 5 minutes such as 2 to 3 minutes. An effective amount of PEG may therefore be titrated accordingly.

The degree of EGFR reduction may be determined using methods described in the examples herein. In particular, the degree of EGFR reduction may be determined using flow cytometry as described below.

The EGFR expression status of a target tissue may be determined using standard methods and kits (for example EGFR pharmDx™, available from Dako Denmark A/S, Glostrup, Denmark).

An effective amount of PEG for use in the prophylaxis of HNSCC may differ from an effective amount of PEG for use in the treatment of HNSCC. In one embodiment, in a prophylaxis setting, a lower amount of PEG is required than typically used in a treatment setting. Since PEG is already widely used in the medical field and is well tolerated, the risk of serious adverse events is considered to be low. Since the present inventors have demonstrated that PEG is capable of inhibiting proliferation of HNSCC cells in vitro in a concentration dependent manner (see below), it is within the purview of the ordinarily skilled person to determine the effective amount of PEG according to the considerations described above.

Compositions of the invention may comprise an effective amount of PEG. For example, aqueous solutions of the invention (such as a mouthwash) may comprise 7.5% w/v or greater (for example 10% w/v or greater) of PEG. Compositions of the invention comprising an effective amount of PEG may be administered one or more times per day, for example, twice per day, three times, four times or five times per day. The duration of therapy with compositions of the invention depends, in part, on the considerations given above. Such considerations are within the purview of the attending physician or healthcare professional.

Therapeutic Agents.

Compositions of present invention comprising an effective amount of PEG may be used in conjunction with one or more therapeutic agents for the prophylaxis and/or treatment of HNSCC. For example, compositions of the invention may be co-administered with the one or more therapeutic agent(s). The term “co-administered” means the coordinated administration of compositions of the invention with one or more therapeutic agents to prevent and/or treat HNSCC. Such coordinated administration between compositions of the invention and one or more therapeutic agent(s) may be simultaneous, sequential or separate.

Examples of therapeutic agents that may be used in conjunction with compositions of the invention include radiation therapy and anti-cancer agents. The term “anti-cancer agent” means a therapeutic agent that is capable of inhibiting the initiation and/or proliferation of cancer and/or promoting cell death (e.g. by apoptosis) in cancer cells such as squamous cell carcinomas, particularly those of the head and neck. Such therapeutic agents include those approved by national or regional regulatory authorities for such use.

Examples of anti-cancer agents include agents that target EGFR expression and/or function (for example by inhibiting functional signaling of the EGFR), herein referred to as “anti-EGFR” agents“. Examples of anti-EGFR agents include anti-EGFR antibodies such as cetixumab, panitumumab, zalutumab, nimotuzumab. Other anti-EGFR agents include Erlotinib, Gefitinib, Lapatinib, BIBW-2992.

Examples of anti-cancer agents include agents that target VEGFR expression and/or function (“anti-VEGFR agents”). Examples of anti-VEGFR agents include bevacizumab, sorafenib, sunitinib, vandetanib.

Examples of anti-cancer agents include agents that target IGF-1R expression and/or function (“anti-IGF-1R agents”). Examples of anti-IGF-1R agents include figitumumab and cixtumumab.

Examples of anti-cancer agents include agents that inhibit the mammalian target of rapamycin (“mTOR agents”). Examples of such mTOR agents include Temsirolimus, Everolimus.

Further examples of anti-cancer agents include platinating agents such as cisplatin and carboplatin; taxanes such as paclitaxel and docetaxel; folate anti-metabolites such as pemetrexed; fluorouracil, methotrexate.

Examples of other anti-cancer agents include Dasatinib, lonafarnib and Bortezomib.

In some embodiments, compositions of the present invention comprise an effective amount of PEG together (for example in intimate physical admixture) with an effective amount of one or more anti-cancer agents such as described above. Preferably, the PEG is not conjugated to the anti-cancer agent. The reader of this specification may assume that each combination of PEG together with one or more of the anti-cancer agents described above is individually and specifically contemplated as an embodiment of the invention.

It will be apparent that an effective amount of one or more therapeutic agent(s) need not necessarily be the same weight amount as an effective amount of PEG. It will also be apparent that when considering the term “effective amount” in relation to radiation therapy, an appropriate dose unit (for example gray (gy) or rad) should be deployed.

In other embodiments of the invention there is provided a kit comprising a composition of the invention together with at least one composition of one or more anti-cancer agents such as described above, optionally together with instructions for use.

Methods of Treatment and Compositions for Use in Such Methods.

The present invention provides a method for preventing and/or treating HNSCC in a subject comprising administering an effective amount of PEG. The present invention also provides compositions for use in the prophylaxis and/or treatment of HNSCC in a subject.

Compositions and treatment methods of the present invention may be of particular use in treating squamous cell carcinomas such as HNSCC that over-express surface EGFR. The EGFR expression status of a squamous cell carcinoma in a subject may be determined according to standard methods and kits (for example EGFR pharmDx™, available from Dako Denmark A/S, Glostrup, Denmark).

In one embodiment, the method is for preventing HNSCC. In another embodiment, the method is for treating HNSCC. In a further embodiment, the method is for preventing and treating HNSCC.

The synonymous terms “prophylaxis” and “preventing” and grammatical variations thereof means inhibiting the initiation of HNSCC (or, as the case may be, cancer of the esophagus) and/or inhibiting the progression of a pre-malignant pathology of the epithelium (such as oral leukoplakia) and/or early stage carcinoma to a later stage carcinoma.

In some embodiments, prophylaxis methods of the invention are performed prior to a positive diagnosis of HNSCC.

The term “treat” and grammatical variations thereof means inhibiting the proliferation of HNSCC (or, as the case may be, cancer of the esophagus) and/or promoting cell death in HNSCC (or, as the case may be, a cancer of the esophagus). In some embodiments, treat includes “cure”, although the term cure does not necessarily mean the complete restoration of health with respect to the malignancy. Those skilled in the art recognize that a treatment may have varying degrees of curative effect and as such are encompassed by the term “treat”.

The present invention further provides a method for preventing HNSCC in a subject in remission of that disease comprising administering to the subject a composition of the invention. The remission may be total or partial. Compositions for use in such a method are also provided.

The present invention further provides a method for treating locally advanced and/or metastatic HNSCC in a subject comprising administering to the subject a composition of the invention.

In another embodiment of the invention there is provided a method for preventing and/or treating HNSCC in a subject susceptible to developing HNSCC comprising administering to the subject a composition of the invention. Such subjects include those with a history and/or concurrent use of: tobacco (smoking and/or chewing), heavy alcohol intake, betel nut chewing. Such subjects include those that are infected with HPV (particularly with serotype HPV 16) and/or immunocompromised and/or with a prior or familial history of (or predisposition to) developing HNSCC. The invention further provides a method for preventing and/or treating HNSCC in a subject that is HPV negative comprising administering to the subject a composition of the invention. Compositions for use in such methods are also provided.

In another embodiment, there is provided a method for preventing HNSCC in a subject afflicted with oral leukoplakia and/or erythroplakia comprising administering to the subject a composition of the invention. Compositions for use in such a method are also provided.

In another embodiment, there is provided a method for preventing HNSCC in a subject afflicted with pre-malignant alterations to the epithelium of the upper aerodigestive tract comprising administering to the subject a composition of the invention. Pre-malignant alterations may arise as a result of “field cancerization”. Field cancerization refers to a process whereby the epithelium undergoes alterations (which may be multiple and independent of one another) that primes the epithelium for transformation. These alterations may be evident in subtle changes to the epithelium vasculature, cellular dysplasia and other molecular changes to the epithelium. This embodiment may be particularly apt in preventing HNSCC in subjects with a prior history of (or predisposition to) HNSCC.

In another aspect of the invention there is provided a method for preventing and/or treating a squamous cell carcinoma such as HNSCC in a subject which method comprises:
(a) administering to said subject an effective amount of a therapeutic agent such as radiation therapy and/or an anti-cancer agent, e.g. an anti-EGFR agent (for example an anti-EGFR antibody such as cetixumab);
(b) administering to said subject an effective amount of PEG.

In one embodiment of this aspect of the invention, step (b) and step (a) occur concurrently. In another embodiment step (b) occurs after step (a). In another embodiment, step (b) occurs before step (a). In another embodiment of this aspect of the invention, the method further comprises the step of (c) administering an effective amount of a therapeutic agent e.g. radiation therapy and/or an anti-cancer agent such as an anti-EGFR agent (for example, an anti-EGFR antibody such as cetixumab). In this embodiment, step (b) may occur after step (a) and before step (c). Therefore, an effective amount of PEG may be administered between cycles of treatment with other therapeutic agents, particularly radiation and/or anti-cancer agents. The therapeutic agents of step (a) and step (c) need not necessarily be the same. Where the therapeutic agents of step (a) and step (c) are the same, the posology followed in step (a) and step (c) need not necessarily be the same.

In another aspect of the invention, an effective amount of PEG may be used to treat HNSCC in a subject in conjunction with resection and/or ablation of a HNSCC carcinoma. Accordingly, there is provided a method for treating HNSCC in a subject comprising the steps of:
(a) resecting and/or ablating at least one HNSCC carcinoma;
(b) administering an effective amount of PEG.

Step (a) may occur either before step (b) or after step (b). In some embodiments of this aspect of the invention, there may be a further step (c) comprising the step of administering an effective amount of a therapeutic agent as described herein. Step (c) may occur after step (a) and before, after or concurrently with step (b).

In some embodiments of this aspect of the invention, an effective amount of PEG may be administered at the site(s) and local region of the surgical resection/ablation e.g. locoregional administration. In some embodiments of the invention, an effective amount of PEG may be administered (e.g. locoregionally administered) to a lesion of the head and neck suspected of being afflicted with HNSCC.

The present invention further provides methods for the multi-modal treatment of HNSCC (that is treatment involving at least two different modalities for treating HNSCC, e.g. surgery, radiotherapy, chemotherapy) the method comprising administering an effective amount of PEG.

In accordance with another embodiment, the present invention provides a method for reducing or inhibiting EGFR expression and/or phosphorylation in the squamous cells of the head and neck of a subject, by administering to the subject an effective amount of PEG. The dosages and duration of use is dependent on the amount of reduction of EGFR expression or phosphorylation desired. Preferably, in accordance with an embodiment of the present invention, PEG is administered to the subject at least 1 to 14 days, however PEG can be administered for a longer period, or until the reduction of expression and/or phosphorylation of EGFR in the squamous cell tissues is achieved.

In accordance with an aspect of the invention there is provided a method for preventing and/or treating cancer of the esophagus in a subject comprising administering to the subject an effective amount of PEG. Compositions, such as described above, for use in such a method are also provided.

In accordance with a further aspect of the invention there is provided a method for reducing the tumor burden (i.e. tumor number and volume) in a subject afflicted with a squamous cell carcinoma such as HNSCC comprising administering an effective amount of PEG. Compositions for use in such as method are also provided.

The subject referred to in the inventive methods can be any subject. Preferably, the subject is a mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is human, such as a male human.

### EXEMPLIFICATION

The following examples further illustrate the invention but should not be construed as in any way limiting its scope.

Animals Studies and Tumor Induction.

All animal protocols were reviewed and approved by the Institutional Animal Care and Use Committee of Northshore University HealthSystem.

4NQO Head and Neck Squamous Cell Carcinogenesis Model:

Twenty-four male Fisher rats (F 344; 150-200 g; Harlan, Indianapolis, Ind.) were housed, three to a plastic cage, in a climate controlled environment (25° C., 60% humidity and a 12 hour light/day cycle). Sixteen animals were provided ad libitum rat chow and drinking water supplemented with 4-nitroquinoline 1-oxide (4NQO; 20 ppm; Sigma Chemicals, St. Louis, Mo.). Freshly prepared 4NQO fortified water was dispensed to rats in colored (light opaque) bottles that were replenished two times a week. The remaining 8 rats were provided clean drinking water (control group). After 14 weeks, the 4NQO supplemented water was replaced with regular water and the rats randomized into two treatment groups. The first group (8 rats) received a daily topical application of PEG-8000 in the oral cavity. This was accomplished by painting a solution of 10% w/v PEG in normal saline, for at least 2 to 3 minutes, on the buccal floor/roof of the rat oral cavity using a sable brush (#4). The second group (8 rats), serving as the control, were painted with normal saline only. This regimen was continued for 14 additional weeks. The rats were periodically examined for the presence of any gross morphological changes on the protruded tongue and the oral cavity. At the end of 14 weeks, the animals were euthanized under controlled CO₂ exposure/bilateral thoracotomy. At necropsy, rat tongues were excised, and subjected to macroscopic tumor assessment. The tongue sections were sliced, formalin fixed, paraffin embedded, sectioned and subjected to histological and immunohistochemical processing.

The dissected rat tongues were examined for the presence of overt tumors. Total number of tumors (>0.2 cm) on each tongue were counted and the tumor volume (size) was measured according to the formula width×length×height×Tr/6 (Tomayko M. M. and Reynolds C. P., Cancer Chemother. Pharmacol., 24:148-154 (1989)). Histological evaluations for the presence of epithelial atypia and dysplasia in the uninvolved tongue tissue were performed after staining with hematoxylin and eosin.

Head and Neck Orthotopic Squamous Cell Carcinoma Model.

In this xenograft model, the squamous cells (SCC 25) were directly implanted near the anatomic site of origin in the oral cavity to mimic local tumor growth. This model allows studying the effect of PEG on the regression of tumor growth. B-cell deficient athymic (nu/nu) mice were procured from Charles River (MA). SCC-25 cells [1×10⁶ cells in 50 μl DMEM F12 media (ATCC)] were directly injected into the upper tongue region of the mice. Two weeks later, when the tumors started appearing, the mice were randomly divided into two groups of 12 animals each and subjected to once-daily oral topical application of 10% w/v PEG 8000 (˜100 μL) or PBS using a Q-tip.

The mice were euthanized 19 to 20 days post treatment under CO₂ exposure/bilateral thoracotomy and tumors excised and weighed. Tumor and tongue tissues were formalin fixed, paraffin embedded/mounted before staining with hematoxylin and eosin. Histopathological evaluation confirmed the squamous nature of the tumors. Also evaluated were the proliferation marker Ki-67 and EGFR by immunostaining of the tongue sections.

Immunohistochemical (IHC) Analysis.

Tissue sections were subjected to IHC analysis to determine the effect of PEG 8000 on the expression of proliferation marker Ki-67 and EGFR. Four micron paraffin-embedded sections were mounted on Superfrost⁺ slides (Vector Laboratories, Burlingame, Calif.) and deparaffinized first by baking at 55-60° C. for 1 hour, and then subjecting to two 5 minute washes in xylene. The tissue sections were then hydrated in graded series of ethanol rinses. The epitope retrieval was performed by subjecting the tissue slides to pressure microwaving (NordicWare, Minneapolis, Minn.) in antigen unmasking solution (Vector Laboratories). Endogenous peroxide activity was quenched by treating with 3% H₂O₂ in methanol for 10 minutes, and the nonspecific binding was blocked by incubating the tissue sections with 5% horse serum for 2 hours at room temperature. Sections were then incubated at 4° C. for 4 h with primary antibodies anti-Ki-67 (RB-9043-P, 1:300 Thermo Scientific UK), and anti-EGFR (SC-03, 1:200 Santa Cruz Biotechnology, Santa Cruz, Calif.), followed by appropriate biotinylated secondary antibodies. After repeated PBS washing, the antigen-antibody complexes were detected with the Vectastatin Elite ABC kit (Vector Laboratories) using 3,3′-diaminobenzidine (DAB) as chromagen (Invitrogen, Calif.). For negative controls, sections were processed in the absence of the primary antibodies. Specimens were counterstained in Gill's hematoxylin solution and the blue color stabilized by a 20 second wash in saturated lithium carbonate (1 g/100 ml). Immunohistochemistry (IHC) was scored by a pathologist with no prior knowledge of the treatment plan. A semiquantitative scale was used to evaluate immunoreactivity of basal squamous epithelial cells. The extent of staining was graded and scored as 0, negative staining; 1+, <10% reactivity, 2+ 10-50% reactive, and 3+ for >50% positive reactivity.

Cell Culture.

SCC-25 and SCC-9 cells (American Type Tissue Culture, Rockville, Md.) were cultured in DMEM/F-12 media (containing 2.5 mM L-glutamine, 15 mM HEPES, 0.5 mM sodium pyruvate, and 1200 mg/l sodium bicarbonate) supplemented with 400 ng/ml of hydrocortisone (Sigma/Adrich), 10% v/v FBS (ATCC), and 0.5% v/v Pen/Strep (ATCC). The OKF-6 (HPV−) and HOK (HPV+) cells were cultures in keratinocytes-SFM media (Life Technologies). To assess the effect of PEG, these cells were treated with various formulations of PEG or vehicle (PBS) for 24 h as described in the examples below. Cells were assessed for cell proliferation (WST-1) or subjected to Western Blot and/or flow cytometric analyses.

Western Blot Analysis.

Western blotting was applied using standard techniques. Briefly, 30 μg protein was subjected to SDS-PAGE, transferred to polyvinylidene difluoride membranes (Amersham Pharmacia, Piscataway, N.J.), blocked with 5% blotto (5% milk, 0.05% tween in TBS buffer) and probed with specific antibodies to proliferating cell nuclear antigen (PCNA, SC-56, 1:1000, Santa Cruz Biotechnology), epidermal growth factor receptor (EGFR, SC-03, 1:750, Santa Cruz Biotechnology), using standard techniques. Xerograms were developed with enhanced chemiluminescence (Santa Cruz Biotechnology). Images were acquired via UVP Bio-imaging Systems and analyzed using Labworks 4.6 software (UVP, LLC, Upland, Calif.). Uniformity in protein loading was achieved by normalization after probing membranes with anti-β-actin (SC-1615 HRP, 1:2000 Santa Cruz Biotechnology).

Cell Proliferation Assay (WST-1).

Cell number was assessed by measuring the cleavage of tetrazolium salt WST-1 (4-[3-(4-iodophenyl)-2-(4-nitrophenyl)-2H-5-tetrazolio]-1, 3-benzene disulfonate) to formazan according to the manufacturer's instructions (Roche Diagnostics, Indianapolis, Ind., USA). Briefly, cells were grown in 96 well plates in a final volume of 100 μL and then incubated with 10 μL of the WST-1 reagent at 37 ° C. for 30 minutes in a humidified 5% CO₂ incubator. Conversion of tetrazolium salt into formazan was determined spectrophotometrically at 440 nm absorbance (Molecular Devices, Sunnyvale, Calif., USA).

Flow Cytometric Analyses.

The following technique was utilized to measure the surface expression of EGFR in SCC-25 and SCC-9 cells treated with PEG. Cells (60-70% confluent) were treated with PEG as described above and then fixed in 4% buffered formaldehyde for 30 minutes. The cells were washed twice in PBS (pH 7.4; containing 2% fetal bovine serum, 0.2% bovine serum albumin and 0.02% sodium azide) followed by incubation at room temperature for 1 hour with anti-EGFR clone 528 (1:200; kindly provided by Dr. H. Band). After washing in PBS, cells were incubated with secondary antibody Alexa 488 Green Fl1-labeled anti-mouse (SC-120AF 488, 1:200, Santa Cruz Biotechnology) for 40 minutes. Cells were subsequently washed in PBS and subjected to flow cytometric analysis (Becton Dickinson Labware, Franklin Lakes, N.J.).

Statistical Analysis.

Data were analyzed by Student's t-test and expressed as means±S.D. or SEM. Differences were considered significant at p<0.05.

### Example 1

This example demonstrates that PEG inhibits cell proliferation in squamous carcinoma cells in vitro.

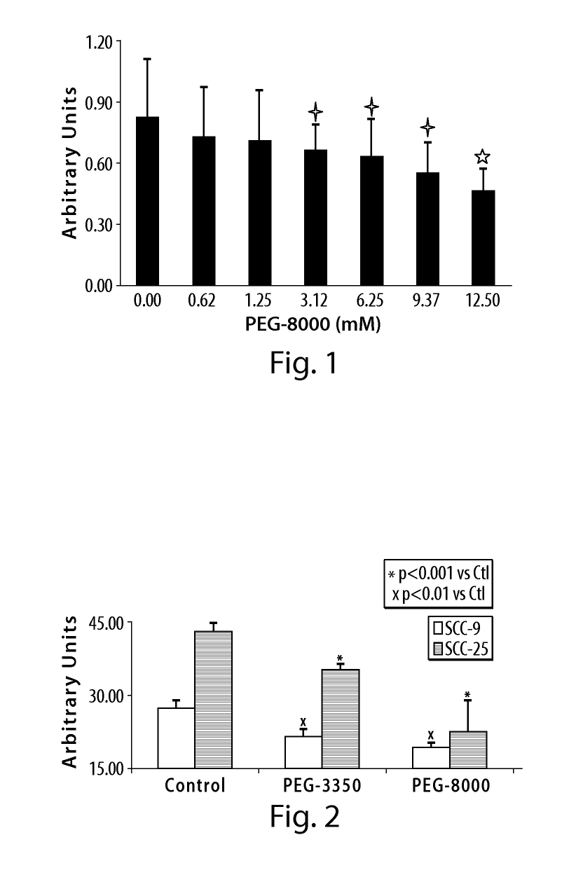

To demonstrate the antiproliferative activity of PEG in HNSCC, SCC-25 cells (2,000 cells/well) were seeded in a 96-well plate and then treated with different concentrations of PEG-8000 [0.62 mM (0.5% w/v) to 12.5 mM (10% w/v)]. After 24 h, the cellular proliferation (changes in cell number) was assayed using the WST-1 assay. As shown in FIG. 1, there was a dose dependent decrease in the SCC-25 cell proliferation ( [symbol] p<0.05; ★p<0.001, compared to vehicle alone). Since a maximal decrease in growth (43% decrease), was obtained with 10% w/v PEG 8000 (the highest concentration tested), this was the dose used for the in vivo experiments. The presence or pretreatment of the SCC-25 cells with PEG significantly reduced cell proliferation.

### Example 2

This example demonstrates that different formulations of PEG inhibit EGFR expression in SCC-9 and SCC-25 cells in vitro.

In these studies, the effect of PEG on the levels of membrane (i.e. surface) EGFR expression in SCC-9 and SCC-25 cells was investigated. Six experimental groups were established. A control group with no treatment for both SCC-9 and SCC-25 cells, a group of SCC-9 and SCC-25 cells treated with a 5% w/v solution of PEG 3350 and a group of SCC-9 and SCC-25 cells treated with a 5% w/v solution of PEG 8000. After 24 h, the cells were trypsinized and subjected to flow cytometric analysis (Becton Dickinson Labware) for membrane (i.e. surface) expression of EGFR. The results indicated that both PEG groups significantly reduced the amount of membrane (i.e. surface) EGFR expression in both SCC-9 and SCC-25 cells (FIG. 2).

### Example 3

This example demonstrates that administration of PEG 8000 inhibits tumor progression in the rat 4NQO model of squamous cell cancer.

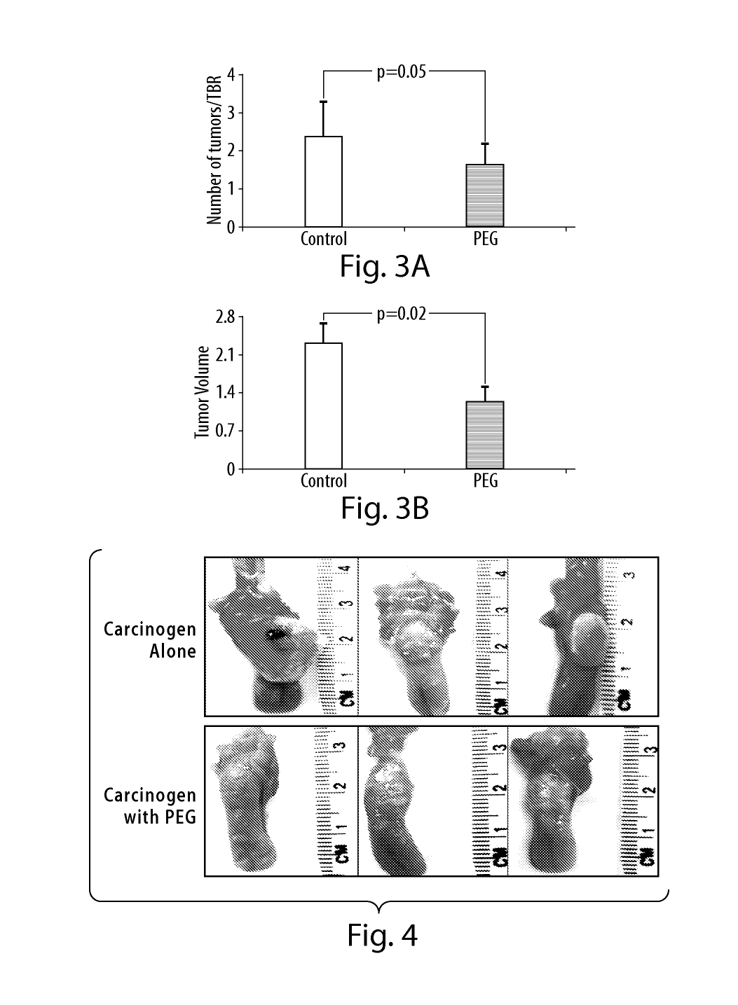

The rat 4NQO-induced oral carcinogenesis model, used in these studies, has a number of features that are histo-pathologically comparable to human head and neck cancer. This model, by systemic administration of 4NQO in drinking water, produces a range of preneoplastic and neoplastic lesions and has been used in number of chemopreventive studies. After 14 weeks of 4-NQO treatment (20 ppm in drinking water), the rats were randomized into two groups, receiving daily oral topical application (painting the buccal floor/roof of rat oral cavity using a sable brush #4 for up to 3-4 minutes) of either (a) vehicle (PBS) or (b) PEG-8000 (10% w/v) for an additional 14 consecutive weeks. At necropsy, the whole tongue, pharynx and esophagus were removed. Overall the carcinogenic regimens used in this study had no debilitating effects on the general health of the rats. As shown in FIG. 3, at necropsy (14 weeks after the end of carcinogen treatment), 4NQO treated rats presented a number of large morphologically discrete lesions or tumors originating from the tongue (p<0.05, compared to control). In contrast, rats subjected to oral topical application of PEG-8000 for 14 weeks post-4NQO treatment presented significantly smaller tumors and fewer tumors/tumor bearing rat (FIG. 3B; p<0.01 compared to control). A photograph comparing the two groups at necropsy is provided in FIG. 4. PEG 8000 did significantly reduced the tumor burden (both tumor number and tumor volume) was significantly diminished. These results imply that PEG 8000 is effective in slowing the initiation and/or progression of cancer in this model.

### Example 4

This example depicts how PEG 8000 inhibits epithelial cell hyperproliferation. Microscopic evaluation of the tongue epithelium from 4NQO treated rats of example 3 revealed a small number of localized regions of mild to moderate epithelial dysplasia.

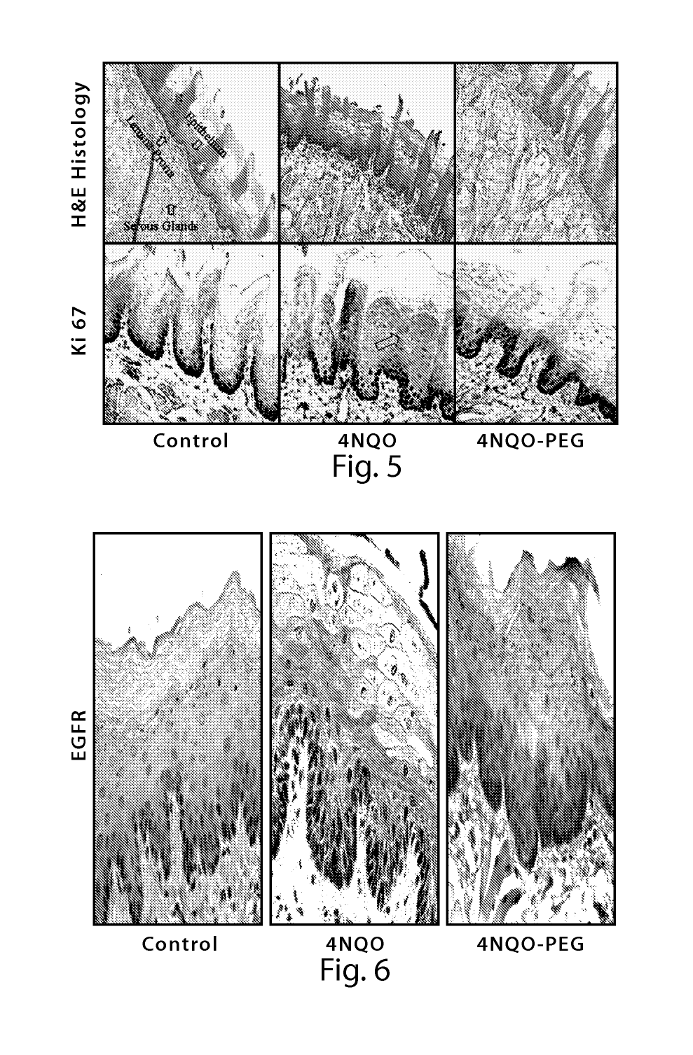

Furthermore, there was a generalized cellular hyperproliferation in the tongue epithelium of the 4NQO-treated rats as has previously been reported in this model (FIG. 5 upper panel). A precise temporal and spatial regulation of the diffused cellular proliferation is an important early characteristic of preneoplastic cancer development, and thus may be an important target for assessing chemopreventive effectiveness of PEG. To understand the antiproliferative role of PEG 8000 in carcinogen treated rats, the immunohistochemical expression of Ki-67, a well-defined marker of proliferation, was examined. The number of Ki-67-labelled epithelial cells (per optical field) was significantly higher in the 4NQO group compared to carcinogen-free control group (30+8 vs. 14+6 respectively; p<0.01, data not shown). However, when the carcinogen-initiated rats were subjected to the topical application of PEG 8000, a significant reduction in the number of Ki-67 positive cells (17+4 vs. 30+8; p<0.01) was observed (data not shown). For this study, a total of 1000 epithelial cells were evaluated in 6 to 7 fields at 400× magnification, and all the values were used for the labeling indices. In the carcinogen treated group, a higher percentage of cells were found to be Ki-67 positive in the supra-basal compartment of the stratified squamous tongue epithelium, when compared to control. PEG 8000 treatment reduced the number of Ki-67 cells in the supra-basal compartment; the remaining Ki-67 cells were restricted mostly to the basal layer as seen in the control group (FIG. 5 lower panel). These results indicate that oral topical application of PEG inhibits epithelial hyperproliferation during 4NQO-induced oral carcinogenesis.

### Example 5

This example illustrates how PEG 8000 down regulates EGFR in the carcinogen initiated rat tongue sections.

The tongues of the rats in Example 3 were sectioned and stained with an antibody to EGFR and subjected to IHC analysis as described above. Treatment with PEG 8000 significantly reduced expression of EGFR as observed on the sections. As illustrated in FIG. 6, the control and 4NQO-PEG sections have little or no staining for EGFR, whereas the 4NQO sections were heavily stained for EGFR. The results show, histologically, that treatment with PEG appears to down-regulate EGFR expression in the rat 4NQO model.

### Example 6

This example illustrates the potential chemopreventive efficacy of topical oral application of PEG 8000 on the development of esophageal tumors.

In addition to studying the chemopreventive benefit of topical PEG 8000 application on the oral carcinogenesis, it was investigated whether PEG 8000 may also provide secondary protection against the development of esophageal tumors. This could be expected due to a partial salivary wash of PEG into the esophagus. For this study the 4-NQO (4-nitroquinoline 1-Oxide) rat model of head and neck cancer was utilized as for example 3, 4 and 5. This model is known to initiate esophageal tumors. The esophagus was very carefully separated from the trachea and subjected to tumor count. The results show that esophageal tumors per tumor bearing rat was decreased from 2.2 in 4 NQO (n=6) to 1.5 in 4 NQO-PEG (n=5) group (˜32% decrease). Although the results did not support statistical significance (p=0.2397—Wilcoxon two sample test; p=0.225—Chi-Square test), the results of this experiment, where contact between PEG and the esophagus was a result of a partial salivary wash rather than a more direct application, nevertheless demonstrates a potential protective effect of PEG against esophageal cancer development.

### Example 7

This example concerns the comparative effect of different PEG formulations on cellular proliferation and EGFR expression in SCC 25 cells.

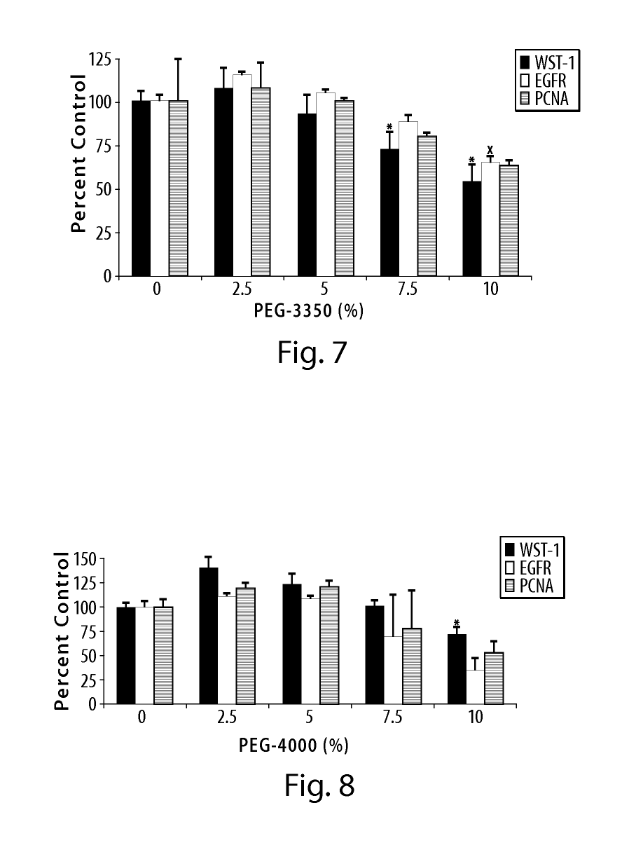

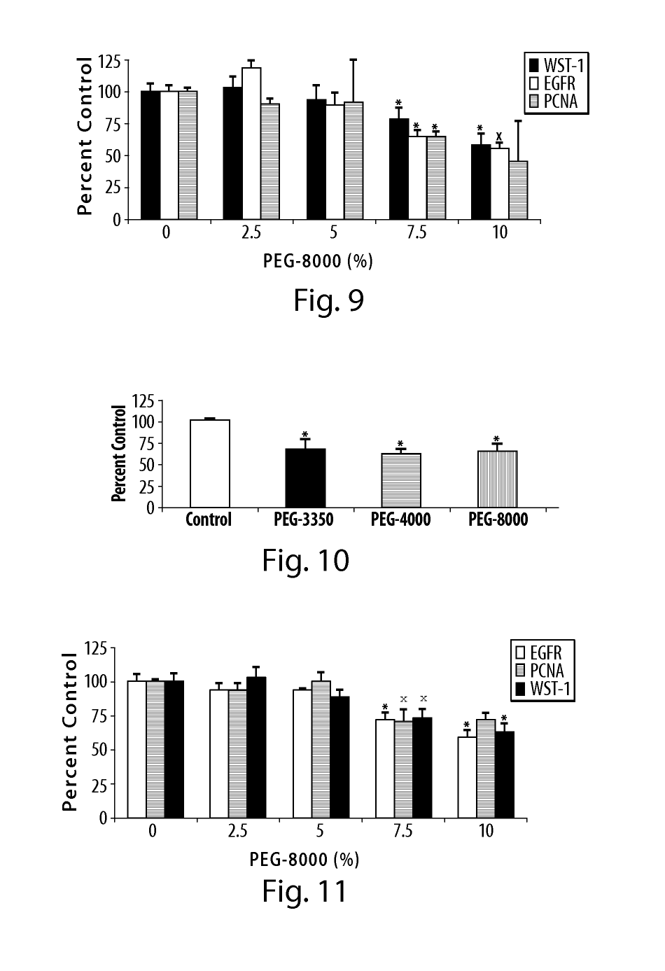

To determine the comparative effect of different PEG formulations on cellular proliferation and EGFR expression, squamous carcinoma cells (SCC-25) were seeded either in 96 well plates (WST-1 assay) or 60 mm cell culture dishes (Western blotting). The cells were then treated for 24 h with PEG 3350, PEG 4000 and PEG 8000 using concentrations ranging from 0 to 10% w/v (FIGS. 7, 8 and 9 respectively). The incubations were carried out in a humidified 5% CO₂ incubator at 37° C. for 24 h. Cellular proliferation (changes in cell number) was assayed using the WST-1 assay as described in Example 1 or via Western blot for the proliferation marker Proliferating Cell Nuclear Antigen (PCNA). EGFR expression was also assessed by Western blot analysis. The results show a linear decrease in cellular proliferation and EGFR expression with increasing concentration of PEG irrespective of the formulation used.

### Example 8

This example illustrates the effect of different PEG formulations on surface expression of EGFR in SCC 9 cells.

These studies were performed to determine the comparative effect of different PEG formulations on the surface expression of EGFR using squamous carcinoma SCC-9 cells. SCC-9 cells were treated for 24 h with 10% w/v PEG (PEG 3350, PEG 4000, or PEG 8000). After treatment, the cells were trypinized and subjected to flow cytometric analysis for membrane (surface) expression of EGFR. The results showed a highly significant decrease in surface expression of EGFR in cells treated with all the PEG formulations tested (FIG. 10). These results further support the hypothesis that the anti-proliferative property of PEG may be related to its anti-EGFR activity.

### Example 9

This example concerns the effect of PEG 8000 on etiologically heterogeneous HNSCC.

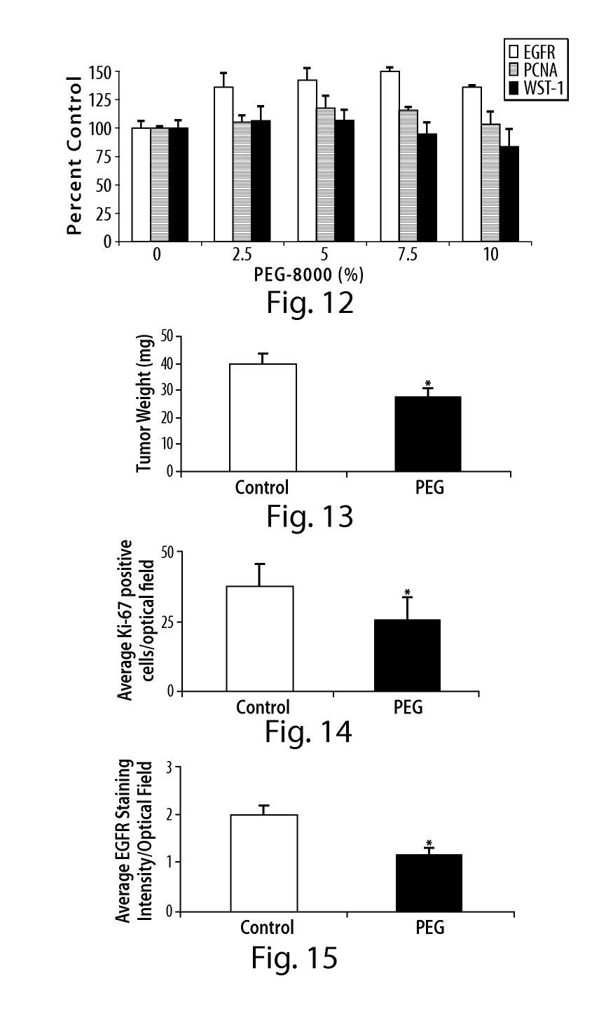

Although tobacco and alcohol consumption are the primary risk factors for the development of HNSCC, it has recently been demonstrated that infection with high-risk human papilloma virus (HPV) may be etiologically linked to development of another subset of HNSCC. In order to study the effect of PEG in etiologically heterogeneous conditions, cell lines were utilized having different HPV status to study the effect of PEG on EGFR down-regulation and cell proliferation. The two cell lines used in these experiments were premalignant hTERT-immortalized cells that are HPV negative [HPV (−) OKF-6; FIG. 11] and premalignant HPV transfected immortalized human oral keratinocytes (HOK) that express E6 [HPV (+) HOK; FIG. 12]. The cells were treated with different concentrations of PEG 8000 for 24 h and subjected to different biomarker assessment as discussed in Examples 1 and 7. The results show that PEG 8000 was effective in significantly reducing the cellular proliferation (WST-1 and PCNA) as well as down-regulating EGFR specifically in HPV (−) cells (FIG. 11) but not in HPV (+) cells (FIG. 12). The differential response to PEG could be attributed to lower expression of EGFR in HPV (+) subset as HPV status has previously been shown to have a significant inverse relationship to EGFR in HNSCC. There is evidence to suggest that the prognosis of HPV (+) subjects may be more favorable compared to HPV (−) subjects. These results indicate that PEG may provide a better strategy for prognostically less favorable HPV (−) subjects.

### Example 10

This example concerns the effect of topical application of PEG 8000 on the development of orthotopic tumors in athymic mice.

In order to study the effect of PEG 8000 on the inhibition of tumor growth, an orthotopic model was utilized in which squamous cell carcinoma (SCC-25, approximately one million) were directly implanted near the tongue region of athymic mice (immuno compromised, B-cell deficient). Two weeks post inoculation, the mice were randomly divided into two groups (12 mice each) and subjected to daily oral topical application of 10% w/v PEG 8000 or PBS (vehicle) using a Q-tip (2-3 min). During the treatment period, PEG 8000 did not cause any significant change in body weights compared to control (data not shown). At necropsy (19-20 days post treatment), the tumors were excised and weighed. The histopathology for all the tumors was positive for squamous cell carcinoma. The results show that PEG 8000 application caused a significant decrease in the tumor weight compared to control (29.8%; p<0.05; FIG. 13). In order to further understand if PEG had an anti-proliferative and anti-EGFR role in the tumor regression, IHC for the markers Ki-67 and EGFR was performed and a significant reduction in both proliferative index (31.5%; p<0.002) and EGFR staining (42.4%; p<0.005) in the PEG 8000 treated groups compared to control was observed (FIGS. 14 and 15 respectively). These results demonstrate that in addition to prevention, PEG appears able to cause regression of an established tumor. This effect may be due to its overall effect on cellular proliferation and EGFR down regulation. This provides a very important tool in further developing a HNSCC treatment strategy.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e. meaning “including, but not limited to”) unless otherwise noted. All methods described herein can be performed in any suitable order unless otherwise indicated herein or contradicted by context. The use of any and all examples, or exemplary language (e.g. “such as”) provided herein, is intended to merely illuminate the invention and does not pose a limitation on the scope of the invention unless otherwise claimed. No language in the specification should be construed as indicating that any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise as specifically described herein. Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

1. A method for preventing and/or treating head and neck squamous cell carcinoma (HNSCC) in a subject comprising administering to the subject an effective amount of polyethylene glycol (PEG).

2. The method of claim 1 wherein the PEG is administered topically.

3. The method of claim 2 wherein the PEG is administered locoregionally to the target tissue.

4. The method of claim 1 wherein the HNSCC has afflicted one or more of: the lip, oral cavity (including the tongue, buccal mucosa, alveolar ridge, retromolar trigone, gums, floor of mouth, hard palate), salivary glands, nasal cavity (including nasopharynx), paranasal sinuses, pharynx (including oropharnyx such as the base of tongue, soft palate, tonsillar pillar and fossa) hypopharnyx (including pyriform sinus, lateral pharyngeal wall, posterior pharyngeal wall, postcricoid pharynx), and larynx (including supraglottis (e.g. false cords, arytenoids, epiglottis, arytenoepiflottic fold), glottis, subglottis) of the subject.

5. The method of claim 4 wherein the HNSCC has afflicted the oral cavity (or anatomical structure thereof).

6. The method of claim 1 wherein the subject is in remission of HNSCC.

7. The method of claim 6 wherein the remission is total or partial.

8. The method of claim 7 wherein the method is to prevent recurrence of HNSCC.

9. The method of claim 1 wherein the HNSCC is locally advanced.

10. The method of claim 1 wherein the HNSCC is metastatic.

11. The method of claim 1 further comprising resecting and/or ablating one or more HNSCC carcinoma.

12. The method of claim 11 comprising the steps of:
(a) resecting and/or ablating one or more HNSCC carcinoma(s);
(b) administering an effective amount of PEG.

13. The method of claim 12 wherein step (a) occurs before step (b).

14. The method of claim 12 wherein step (b) occurs before step (a).

15. The method of claim 1 wherein the PEG is co-administered with one or more therapeutic agent(s).

16. The method of claim 15 wherein the therapeutic agent is radiation therapy and/or an anti-cancer agent.

17. The method of claim 15 wherein the PEG is administered concurrently with the one or more therapeutic agent(s).

18. The method of claim 15 wherein the PEG is administered before administration with the one or more therapeutic agent(s).

19. The method of claim 15 wherein the PEG is administered after administration of the one or more therapeutic agent(s).

20. The method of claim 15 wherein the PEG is administered following a first administration with the one or more therapeutic agent(s) and before a second administration with the one or more therapeutic agents.

21. The method of claim 20 wherein the one or more therapeutic agent(s) of the first administration is the same as the one or more therapeutic agent(s) of the second administration.

22. The method of claim 20 wherein the one or more therapeutic agent(s) of the first administration is different from the one or more therapeutic agent(s) of the second administration.

23. (canceled)

24. (canceled)

25. The method of claim 16 wherein the anti-cancer agent is selected from the group consisting of: anti-EGFR agents, anti-VEGFR agents, anti-IGF-1R agents, mTOR agents, platinating agents, taxanes, folate anti-metabolites, fluorouracil, methotrexate.

26. The method of claim 25 wherein the anti-EGFR agent is an anti-EGFR antibody such as cetixumab.

27. The method of claim 1 wherein the subject is susceptible to developing HNSCC.

28. The method of claim 27 wherein the susceptible subject has a history and/or concurrent use of one or more of the following: tobacco (smoking and/or chewing), heavy alcohol intake, betel nut chewing.

29. The method of claim 27 wherein the subject is HPV infected and/or immunocompromised and/or with a familial or prior history of developing HNSCC.

30. The method of claim 27 wherein the patient is afflicted with oral leukoplakia and/or erythoplakia.

31. The method of claim 1 wherein the subject is a mammal.

32. The method of claim 31 wherein the mammal is a human.

33. A composition for preventing and/or treating head and neck squamous cell carcinoma (HNSCC) in a subject comprising an effective amount of polyethylene glycol (PEG).

34. The composition of claim 33 wherein the PEG has an average molecular weight (for example a weight average molecular weight), in a range wherein the lower limit of the range is selected from the group consisting of: 1000, 2000, 3000, 4000, 6000; and an upper limit of the range is, selected independently, from the group consisting of; 2000, 3000, 4000, 5000, 6000, 7000, 8000, 9000, 10,000, 12,000, 15,000, 20,000.

35. The composition of claim 34 wherein the lower limit is 3000 or 4000 and the upper limit is, selected independently, 5000, 6000, 7000, 8000 or 9000.

36. The composition of claim 35 wherein the PEG has a weight average molecular weight of 3350, 4000, 8000.

37. (canceled)

38. The composition of claim 33 wherein PEG is its sole therapeutically active constituent.

39. The composition of claim 33 further comprising an effective amount of one or more therapeutic agents.

40. The composition of claim 33 further comprising at least one excipient selected from the group: at least one electrolyte, one or more sweetener(s), one or more flavouring(s), a pharmaceutically acceptable carrier.

41. The composition of claim 33 wherein the composition is prepared in the form of: an aqueous solution or suspension (for example a mouthwash), emulsion, paste (e.g. toothpaste), cream, balm (e.g. lip balm), ointment, foam, sponge, gel, chewing gum, spray, lozenge, troche, syrup (e.g. viscous syrup), linctus, slurry, film (e.g. orodispersible film), tablet (e.g. orodispersible tablet), capsule, caplet, buccal patch, granule.

42. (canceled)

43. (canceled)
